(19) United States
(12) Patent Application Publication
(10) Pub. No.: US 2026/0192458 A1
(43) Pub. Date: Jul. 9, 2026

(54) CONVEYANCE SYSTEM

(71) Applicant: SHIMADZU CORPORATION, Kyoto-shi (JP)

(72) Inventors: Tomotaka NAGASHIMA, Kyoto-shi (JP); Ayako AKAZAWA, Kyoto-shi (JP); Kanako MITA, Kyoto-shi (JP); Kohei HARA, Kyoto-shi (JP); Yuka UEDA, Kyoto-shi (JP); Akane KITAGAWA, Kyoto-shi (JP); Masafumi FURUTA, Kyoto-shi (JP); Kenta CHINOMI, Kyoto-shi (JP)

(73) Assignee: SHIMADZU CORPORATION, Kyoto-shi (JP)

(21) Appl. No.: 19/437,438

(22) Filed: Dec. 31, 2025

(30) Foreign Application Priority Data
Jan. 8, 2025 (JP) 2025-002978

(51) Int. Cl. B25J 9/16

(52) U.S. Cl. CPC B25J 9/1694

(57) ABSTRACT

A conveyance system comprises a measurement device configured to measure biological information of a subject, a conveyance mechanism configured to convey the measurement device, a storage device configured to store the biological information of the subject measured using the measurement device conveyed by the conveyance mechanism, a camera interface and a communication interface configured to acquire a status of the subject, and an arithmetic unit configured to control the conveyance mechanism. The arithmetic unit determines whether measurement of the subject's biological information using the measurement device is possible based on the status of the subject, and when the measurement of the subject's biological information is possible, controls the conveyance mechanism to convey the measurement device to the subject.

### TECHNICAL FIELD

The present disclosure relates to a conveyance system.

### BACKGROUND ART

In organizations such as companies, measurement devices that measure the biological information of employees may be introduced to improve their lifestyle habits, maintain health, and reduce disease risks. “Health and Productivity Management” has become a keyword in business operations, and the introduction of such measurement devices in-house is being actively considered. For example, Patent Literature 1 discloses a robot that controls an arm equipped with a measuring instrument for a subject's vital signs to bring the measuring instrument closer to the subject. Patent Literature 2 discloses a robot that prompts a subject to measure their vital signs when it finds a subject whose vital signs have not been measured for a certain period since the last measurement.

### CITATION LIST

### Patent Literature

[Patent Literature 1] Japanese Unexamined Patent Application Publication No. 2020-121353
[Patent Literature 2] Japanese Unexamined Patent Application Publication No. 2022-156958

### SUMMARY OF INVENTION

### Technical Problem

According to the technologies disclosed in Patent Literature 1 and Patent Literature 2, it is possible for a robot to prompt a subject to measure their biological information. However, even if the robot finds a subject to prompt for biological information measurement, the subject may not be in a state where they can measure their biological information, for example, if they are in the middle of work or a meeting.

The present disclosure has been made to solve such a problem, and its objective is to provide a technology capable of conveying a measurement device to a subject at an appropriate time when the subject is able to measure their biological information.

### Solution to Problem

A conveyance system according to an aspect of the present disclosure comprises a measurement device configured to measure biological information of a subject, a conveyance unit configured to convey the measurement device, a storage unit configured to store the biological information of the subject measured using the measurement device conveyed by the conveyance unit, a status acquisition unit configured to acquire a status of the subject, and a control unit configured to control the conveyance unit. The control unit determines whether measurement of the subject's biological information using the measurement device is possible based on the status of the subject acquired by the status acquisition unit, and when the measurement of the subject's biological information is possible, controls the conveyance unit to convey the measurement device to the subject.

A conveyance system according to another aspect of the present disclosure comprises a conveyance unit configured to convey a conveyed object used by a user, a status acquisition unit configured to acquire a status of the user, and a control unit configured to control the conveyance unit. The control unit determines whether conveyance of the conveyed object to the user is possible based on the status of the user acquired by the status acquisition unit, and when the conveyance of the conveyed object is possible, controls the conveyance unit to convey the conveyed object to the user.

### Advantageous Effects of Invention

According to the present disclosure, it is possible to convey the measurement device to the subject at an appropriate time when the subject is able to measure their biological information, based on the status of the subject.

### BRIEF DESCRIPTION OF DRAWINGS

FIG. 1 is a diagram illustrating an application example of a conveyance system according to Embodiment 1.

FIG. 2 is a diagram for explaining the configuration of the conveyance system according to Embodiment 1.

FIG. 3 is a diagram showing a history information table that stores history information of a subject.

FIG. 4 is a flowchart of the main processing executed by a conveyance apparatus.

FIG. 5 is a diagram for explaining the configuration of a conveyance system according to Embodiment 2.

### DESCRIPTION OF EMBODIMENTS

The present embodiment will be described in detail with reference to the drawings. It should be noted that the same or corresponding parts in the drawings are denoted by the same reference signs, and their description will not be repeated in principle.

### Embodiment 1

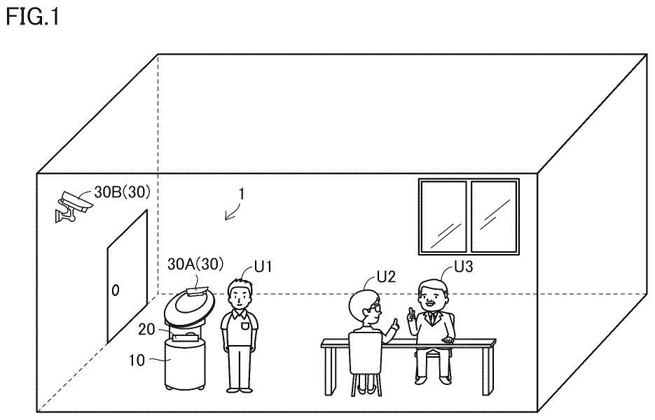

A conveyance system 1 according to Embodiment 1 will be described with reference to FIGS. 1 to 4.

### Application Example of Conveyance System

FIG. 1 is a diagram illustrating an application example of the conveyance system 1 according to Embodiment 1. As shown in FIG. 1, the conveyance system 1 includes a conveyance apparatus 10, a measurement device 20, and cameras 30A and 30B.

The conveyance apparatus 10 is an apparatus (e.g., a robot) that conveys the measurement device 20 to a subject (e.g., a user of the conveyance system 1). The measurement device 20 includes devices capable of measuring a subject's biological information, such as, for example, an AGEs (Advanced Glycation End Products) sensor, a blood pressure monitor, a blood glucose meter, a weight scale, or a body composition analyzer. The camera 30A is installed in a room, such as an office or a facility, and captures images of subjects present in the room. The camera 30B is attached to the conveyance apparatus 10 and captures images of subjects present in the line of sight of the conveyance apparatus 10. Such a conveyance system 1 is introduced into an office or facility to measure the biological information of subjects such as employees or residents. In the example of FIG. 1, the conveyance system 1 is introduced into an office where a plurality of employees, subjects U1, U2, and U3, are present.

AGEs (Advanced Glycation End Products) is a general term for a plurality of compounds, which are produced by the reaction of sugars and proteins through oxidation, dehydration, and condensation. It is believed that AGEs accumulate in the body due to lifestyle irregularities and cause age-related diseases and lifestyle-related diseases (e.g., diabetes, dementia). Therefore, by periodically measuring AGEs, subjects can grasp their health status leading up to a disease in a state where they have not yet developed dementia or hypertension, and can take preventive measures against diseases. Even if a disease develops, the subject can grasp the progression of the disease by periodically measuring AGEs. Although there are individual differences, AGEs generally change in about one to several weeks, so it is desirable for the subject to measure AGEs every week.

However, even if a measurement device 20 such as an AGEs sensor is introduced into an office, not all subjects may show interest and actively use the measurement device 20. The existence of the measurement device 20 may be forgotten, or the need for the subject to move to the installation location of the measurement device 20 may become a factor hindering the continuous use of the measurement device 20.

Therefore, in the conveyance system 1 according to Embodiment 1, the conveyance apparatus 10 is configured to determine for each of a plurality of subjects whether it is necessary to measure biological information, and when it is determined that it is necessary to measure the subject's biological information, to identify the subject's location where the subject targeted for biological information measurement is located, and to convey the measurement device 20 to the subject's location. Furthermore, even when the measurement device 20 is conveyed to the subject, the subject is not always in a state where they can measure their biological information, for example, if they are in the middle of work or a meeting. Therefore, the conveyance apparatus 10 is configured to convey the measurement device 20 to the subject at an appropriate time when the subject is able to measure their biological information.

Specifically, the conveyance apparatus 10 stores the elapsed time since the last measurement of biological information for each of a plurality of subjects. The conveyance apparatus 10 selects an arbitrary subject from the plurality of subjects and determines whether the elapsed time during which the selected subject has not measured their biological information has exceeded a predetermined determination time. When the conveyance apparatus 10 determines that the elapsed time during which the subject has not measured their biological information has exceeded the determination time, it determines that it is necessary to measure the subject's biological information. Note that the “elapsed time” is not limited to units of time, but may also be in units of days, months, and years. That is, “elapsed time” is a concept that includes elapsed days, elapsed months, and elapsed years. Similarly, the “determination time” is also a concept that includes determination days, determination months, and determination years, corresponding to the “elapsed time”. The determination time is, for example, a desirable interval for measuring biological information, and in the case of the aforementioned AGEs, it may be set to one week. Further, the conveyance apparatus 10 can identify the elapsed time since the last measurement of biological information based on the subject's biological information measurement history. The elapsed time since the last measurement of the subject's biological information and the measurement history of the biological information are included in history information 132, which will be described later and is stored by the conveyance apparatus 10.

The conveyance apparatus 10 can identify the position of each of the plurality of subjects U1, U2, and U3 based on the captured image of the room obtained by the camera 30A. Further, the conveyance apparatus 10 can capture images in its line of sight with the camera 30B while moving and identify the position of each of the plurality of subjects U1, U2, and U3 based on the obtained captured images. Therefore, when the conveyance apparatus 10 determines that it is necessary to measure the biological information of an arbitrary subject, it can identify the subject's location of the said arbitrary subject based on the captured images of the plurality of subjects U1, U2, and U3 obtained by the camera 30A or the camera 30B.

Note that the conveyance apparatus 10 is not limited to the captured images of the subject and may identify the subject's location based on position information using GPS (Global Positioning System). Alternatively, the conveyance apparatus 10 may identify the subject's location based on the subject's seat information. For example, if a free-address seating system is introduced in an office, the conveyance apparatus 10 may acquire seat information indicating the position of the seat selected by the subject to identify the subject's location. The conveyance apparatus 10 may identify the subject's location based on at least one of the subject's captured image, the subject's position information, and the seat information set for the subject.

Furthermore, the conveyance apparatus 10 is not limited to the location where the subject is present, but may also identify a location near the place where the subject is present (e.g., the entrance of a conference room) or a place where the subject is frequently present (e.g., a break room) based on the subject's captured image or the subject's behavior history, and convey the measurement device 20 to that location.

Furthermore, when the conveyance apparatus 10 has conveyed the measurement device 20 to the subject's location, it outputs a sound (e.g., a beep sound, a speech sound) to prompt the subject to measure their biological information using the measurement device 20. For example, the conveyance apparatus 10 outputs a sound that effectively prompts the subject to measure their biological information using the measurement device 20, according to the subject's personality or preferences. The conveyance apparatus 10 may acquire the subject's personality or preferences from the results of a questionnaire conducted on the subject in advance, or may acquire the subject's personality or preferences by analyzing the history of actions taken toward the subject in the past.

Also, when the conveyance apparatus 10 has conveyed the measurement device 20 to the location of the subject targeted for biological information measurement, it may perform an action to prompt the subject to measure their biological information using the measurement device 20. Here, the method of calling out that is easily accepted differs depending on the subject. Some subjects may wish to actively communicate with the conveyance apparatus 10, such as by being taught how to use the measurement device 20 or what items the measurement device 20 can measure, while other subjects may feel uncomfortable with active communication from the conveyance apparatus 10. Changing behavior according to the subject's status or personality is effective in prompting the measurement of biological information.

Therefore, the conveyance apparatus 10 acts to effectively prompt the subject to measure their biological information using the measurement device 20, according to the subject's personality or preferences. More specifically, for a subject who is prompted to measure biological information by proactive actions such as being touched or spoken to (e.g., a subject who is susceptible to persuasion), the conveyance apparatus 10 performs proactive actions. On the other hand, for a subject who is prompted to measure biological information by passive actions such as moving subtly around the subject, the conveyance apparatus 10 performs passive actions. In this way, the conveyance apparatus 10 operates to attract the subject's interest according to the subject's preferences, personality, or tastes, thereby prompting the subject to measure their biological information. The conveyance apparatus 10 may acquire the subject's preferences, personality, or tastes from the results of a questionnaire conducted on the subject in advance, or may identify the subject's preferences, personality, or tastes by analyzing the history of actions taken by the subject in the past.

Furthermore, when the conveyance apparatus 10 determines that it is necessary to measure the biological information of an arbitrary subject, it may determine whether the subject's current status is an appropriate status for the subject to measure their biological information. Then, when the conveyance apparatus 10 determines that the subject's current status is an appropriate status, it may convey the measurement device 20 to the subject's location.

The timing at which each subject is likely to accept a prompt to use the measurement device 20 differs. Especially in an office, the daily schedule varies from subject to subject, and there is a risk that a prompt to use the measurement device 20 may interfere with their work. Therefore, it is effective for the use of the measurement device 20 that the conveyance apparatus 10 grasps the current status of each subject and conveys the measurement device 20 at a time when it is likely to be accepted.

For example, in cases where there is a high probability that the subject will not measure their biological information even if the measurement device 20 is conveyed, such as when the subject is in a meeting, in a conversation with others, at work, on the phone, or asleep, the conveyance apparatus 10 may be configured not to convey the measurement device 20 to the subject's location. Furthermore, a situation where the subject is wearing headphones or earphones is also not considered an appropriate situation for measuring the subject's biological information. Therefore, the conveyance apparatus 10 may identify the state of the subject based on the subject's captured image, and if the subject is wearing headphones or earphones, determine that the subject's status is not an appropriate status and not convey the measurement device 20 to the subject. On the other hand, in situations other than those mentioned above, such as when the subject is on a break, where there is a high probability that the subject will measure their biological information if the measurement device 20 is conveyed, the conveyance apparatus 10 may convey the measurement device 20 to the subject's location.

The conveyance apparatus 10 may recognize the subject's current status as described above based on the captured image of the subject obtained by the camera 30A or 30B, and determine whether the subject's current status is an appropriate status for measuring the subject's biological information. The conveyance apparatus 10 may also recognize the subject's current status as described above based on a pre-acquired schedule of the subject, and determine whether the subject's current status is an appropriate status. For example, the conveyance apparatus 10 may determine whether the current time is the subject's free time based on the subject's schedule, and convey the measurement device 20 to the subject if the current time is free time. The subject's schedule is included in schedule information 133, which will be described later and is stored by the conveyance apparatus 10.

Furthermore, the schedule information 133 may include, for example, the subject's PC (Personal Computer) usage history acquired from a schedule management tool such as OFFICE 365 (registered trademark). Based on the schedule information 133, if the conveyance apparatus 10 predicts that the subject's PC usage will continue for a predetermined time (e.g., 30 minutes) or more, it may stop the conveyance of the measurement device 20 for that predetermined time, or convey a measurement device 20 that can be measured even while the subject is using the PC. For example, if measuring AGEs is difficult while using a PC, the conveyance apparatus 10 may convey a measurement device 20 for measuring blood pressure by attaching it to the ankle to the subject. In this way, the conveyance apparatus 10 may determine whether the measurement of the subject's biological information is possible by considering the combination of the current time, the content of the subject's work, the subject's location, and the type of measurement device 20 (type corresponding to the required measurement time and the constraint conditions of the measurement site), and convey an appropriate measurement device 20 according to the subject.

In the example of FIG. 1, the conveyance apparatus 10 determines that it is time for subject U1, selected from among the plurality of subjects U1, U2, and U3, to measure their biological information, and conveys the measurement device 20 to subject U1. As a result, even if subject U1 has forgotten that it is time to measure their biological information, the measurement device 20 is conveyed to the location of subject U1 by the conveyance apparatus 10, and the presence of the measurement device 20 near the subject is emphasized. This allows subject U1 to avoid missing the timing for measuring their biological information without forgetting to do so. Also, even if subject U1 is not interested in measuring their biological information, they can become interested in measuring it by having the measurement device 20 conveyed to their location by the conveyance apparatus 10. Furthermore, since subject U1 does not need to go to the installation location of the measurement device 20, it becomes easier for them to use the measurement device 20 continuously.

Also, among the plurality of subjects U1, U2, and U3, the conveyance apparatus 10 does not convey the measurement device 20 to subjects U2 and U3 because they are in a conversation, even if it is time for subjects U2 and U3 to measure their biological information. This allows the conveyance apparatus 10 to increase the probability that the subject to whom the measurement device 20 is conveyed will measure their biological information using the measurement device 20, and to avoid unnecessary conveyance.

### Configuration of Conveyance System

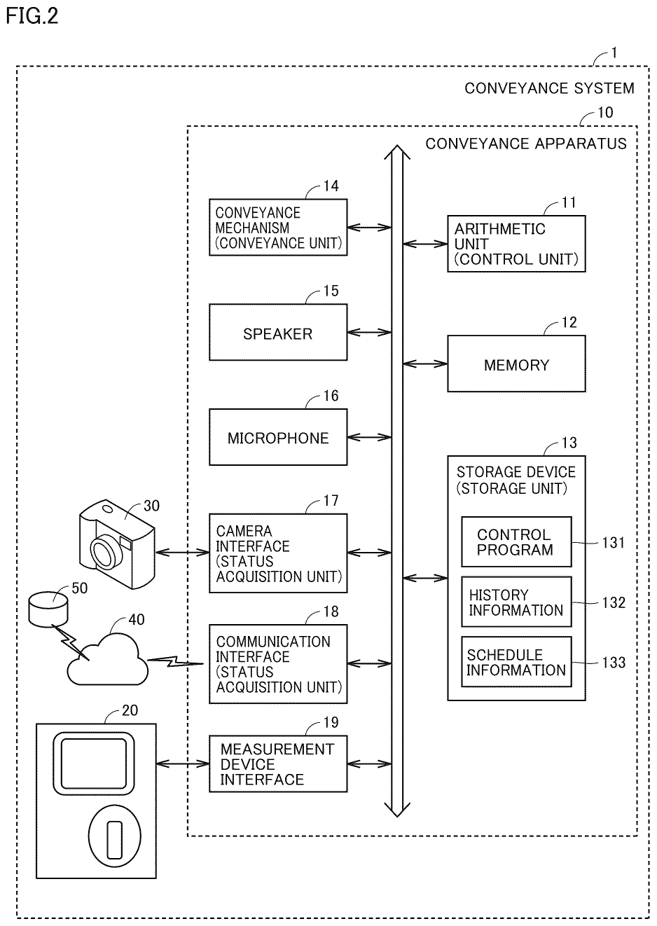

FIG. 2 is a diagram for explaining the configuration of the conveyance system 1 according to Embodiment 1. As shown in FIG. 2, the conveyance apparatus 10 includes an arithmetic unit 11, a memory 12, a storage device 13, a conveyance mechanism 14, a speaker 15, a microphone 16, a camera interface 17, a communication interface 18, and a measurement device interface 19.

The arithmetic unit 11 is an example of a “control unit” and is a computing entity (computer) that executes predetermined processing. The arithmetic unit 11 is composed of a processor such as a CPU (Central Processing Unit), MPU (Micro-Processing Unit), TPU (Tensor Processing Unit), or GPU (Graphics Processing Unit). It should be noted that a processor, which is an example of the arithmetic unit 11, has a function of executing predetermined processing by executing a predetermined program, but some or all of these functions may be implemented using dedicated hardware circuits such as an ASIC (Application Specific Integrated Circuit) or an FPGA (Field-Programmable Gate Array). “Processor” is not limited to a narrow sense processor that executes processing in a stored-program manner like a CPU, MPU, TPU, or GPU, but may include hard-wired circuits such as an ASIC or FPGA. The arithmetic unit 11 as described above can also be read as a processing circuitry. Note that the arithmetic unit 11 may be configured with a single chip or a plurality of chips. Furthermore, the processor and related processing circuits may be configured with a plurality of computers interconnected by wire or wirelessly via a local area network or a wireless network. The processor and related processing circuits may be configured as a cloud computer that remotely performs calculations based on input data and outputs the calculation results to other devices at a remote location.

The memory 12 includes a volatile storage area (e.g., a working area) that temporarily stores program codes or work memory when the arithmetic unit 11 executes various programs. Examples of the memory 12 include volatile memories such as DRAM (Dynamic Random Access Memory) and SRAM (Static Random Access Memory), or non-volatile memories such as ROM (Read Only Memory) and flash memory.

The storage device 13 is an example of a “storage unit” and stores various programs executed by the arithmetic unit 11, various data, and the like. For example, the storage device 13 stores a control program 131 executed by the arithmetic unit 11, and history information 132 and schedule information 133 used in processing according to the control program 131. Furthermore, the storage device 13 stores the biological information of the subject measured using the measurement device 20. Note that the biological information of the subject acquired by the conveyance apparatus 10 may be output from the conveyance apparatus 10 to an external device 50 and stored by the external device 50. The storage device 13 may be one or more non-transitory computer readable media or one or more computer readable storage media. Examples of the storage device 13 include an HDD (Hard Disk Drive) and an SSD (Solid State Drive).

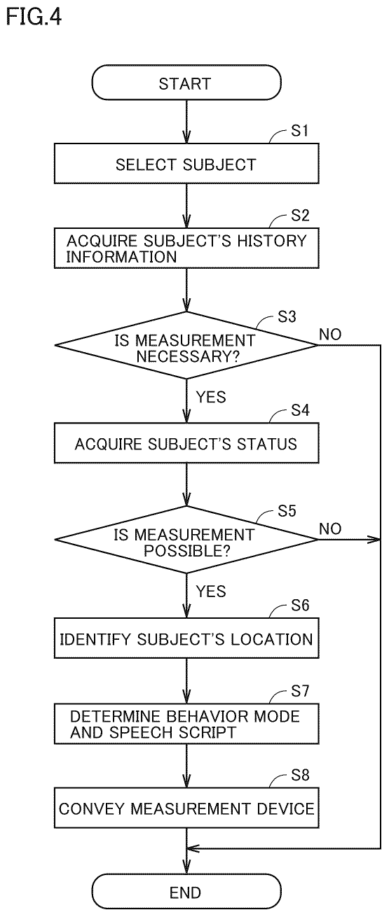

The control program 131 includes a program in which a processing procedure for the arithmetic unit 11 to execute a conveyance process for conveying the measurement device 20 to the subject is defined. Details of the conveyance process will be described later with reference to FIG. 4. By executing the conveyance process according to the control program 131, the arithmetic unit 11 can convey the measurement device 20 to a subject who needs to measure their biological information using the measurement device 20. The control program 131 may be stored in the storage device 13 in advance, or may be acquired from the external device 50 by communicating with it.

The history information 132 includes information regarding the measurement history of the subject's biological information. The schedule information 133 includes information regarding the subject's schedule. The history information 132 and the schedule information 133 may be stored in the storage device 13 in advance, or may be acquired from the external device 50 by communicating with it.

The conveyance mechanism 14 is an example of a “conveyance unit” and includes mechanisms such as a motor necessary for the conveyance apparatus 10 to operate. The conveyance apparatus 10 can convey the measurement device 20 to the subject by the conveyance mechanism 14 being driven under the control of the arithmetic unit 11. The term “conveyance unit” may also refer to the entire conveyance apparatus 10.

The speaker 15 outputs a sound (e.g., a beep sound, a speech sound) to prompt the subject to measure their biological information using the measurement device 20.

The microphone 16 acquires sound such as the subject's voice. The conveyance apparatus 10 can operate according to the subject's instructions by recognizing the subject's voice acquired by the microphone 16. For example, if the subject to whom the measurement device 20 was conveyed utters a voice indicating refusal to measure their biological information, the conveyance apparatus 10 may recognize that voice and act to move away from the subject.

The camera interface 17 is an interface for communicating with the camera 30. The camera 30 includes the camera 30A installed in a room such as an office or facility, as shown in FIG. 1, and the camera 30B attached to the conveyance apparatus 10. The conveyance apparatus 10 can acquire data of a captured image obtained by shooting with the camera 30 via the camera interface 17. The camera interface 17 can be an example of a “status acquisition unit” for the arithmetic unit 11 to acquire a captured image for identifying the subject's status.

The communication interface 18 is an interface for communicating with an external device 50 via a network 40 through a wired or wireless connection. For example, the conveyance apparatus 10 can acquire data such as the control program 131, the history information 132, and the schedule information 133 from the external device 50 by communicating with the external device 50 via the communication interface 18 and the network 40. The communication interface 18 can be an example of a “status acquisition unit” for the arithmetic unit 11 to acquire the schedule information 133 for identifying the subject's status.

The measurement device interface 19 is an interface for communicating with the measurement device 20. For example, the conveyance apparatus 10 can acquire the measurement results of the subject's biological information measured by the measurement device 20 by communicating with the measurement device 20 via the measurement device interface 19. Note that the conveyance apparatus 10 is not limited to communicating directly with the measurement device 20, but may also acquire the measurement results of biological information from the external device 50 via the communication interface 18.

### History Information Table

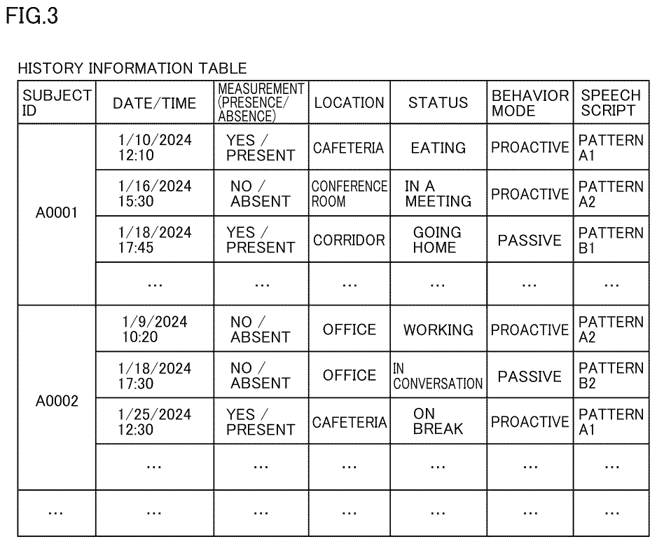

FIG. 3 is a diagram showing a history information table that stores the history information 132 of a subject. As shown in FIG. 3, the history information table stores subject ID information, date and time information, measurement presence/absence information, location information, status information, behavior mode information, and speech script information.

The subject ID information includes a subject ID assigned in advance to each subject for identification. In the history information table, history information such as date and time is stored in association with the subject ID.

The date and time information indicates the date (year, month, day) and time when the conveyance apparatus 10 conveyed the measurement device 20 to the subject. The date and time information includes not only the date and time when the subject measured their biological information using the measurement device 20 conveyed by the conveyance apparatus 10, but also the date and time when the conveyance apparatus 10 conveyed the measurement device 20 to the subject but the subject did not measure their biological information using the said measurement device 20. Based on the date and time information, the conveyance apparatus 10 can identify the date and time when the measurement device 20 was conveyed to the subject in the past. Thus, the history information 132 includes the elapsed time since the subject last measured their biological information.

The measurement presence/absence information indicates whether the subject measured their biological information using the measurement device 20 conveyed by the conveyance apparatus 10. Based on the measurement presence/absence information, the conveyance apparatus 10 can identify whether the subject measured their biological information using the said measurement device 20 when the measurement device 20 was conveyed to the subject in the past.

The location information indicates the location where the conveyance apparatus 10 conveyed the measurement device 20 to the subject. Based on the location information, the conveyance apparatus 10 can identify the location where the measurement device 20 was conveyed to the subject in the past. In the example of FIG. 3, information such as a cafeteria, a conference room, a corridor, and an office is included in the location information as places where the conveyance apparatus 10 conveyed the measurement device 20 among the places where the subject could be present.

The status information indicates the status of the subject when the conveyance apparatus 10 conveyed the measurement device 20 to the subject. Based on the status information, the conveyance apparatus 10 can identify the status of the subject when the measurement device 20 was conveyed to the subject in the past. In the example of FIG. 3, information such as “eating,” “in a meeting,” “going home,” “in conversation,” and “on break” is included in the status information as the subject's status.

The behavior mode information indicates the behavior mode taken by the conveyance apparatus 10 when it conveyed the measurement device 20 to the subject. Based on the behavior mode information, the conveyance apparatus 10 can identify the behavior mode taken when the measurement device 20 was conveyed to the subject in the past. In the example of FIG. 3, a proactive behavior mode and a passive behavior mode of the conveyance apparatus 10 are included in the behavior mode information. For example, when the conveyance apparatus 10 takes a proactive behavior mode, it touches or speaks to the subject to whom the measurement device 20 was conveyed. When the conveyance apparatus 10 takes a passive behavior mode, it moves subtly around the subject to whom the measurement device 20 was conveyed.

The speech script information indicates the content of the speech sound uttered by the conveyance apparatus 10 when it conveyed the measurement device 20 to the subject. Based on the speech script information, the conveyance apparatus 10 can identify the content of the speech sound uttered when the measurement device 20 was conveyed to the subject in the past. In the example of FIG. 3, patterns A1 and A2, which are speech scripts corresponding to a proactive behavior mode, and patterns B1 and B2, which are speech scripts corresponding to a passive behavior mode, are included in the speech script information.

By analyzing the past behavior history of the subject based on the history information stored in the history information table as described above, the conveyance apparatus 10 can identify the preferences, personality, or tastes of each subject. This allows the conveyance apparatus 10 to determine the optimal location, status, behavior mode, and speech script to increase the probability that the subject will measure their biological information using the measurement device 20.

### Processing of Conveyance Apparatus

FIG. 4 is a flowchart of the main processing executed by the conveyance apparatus 10. The processing steps of the conveyance apparatus 10 shown in FIG. 4 are realized by the arithmetic unit 11 executing the control program 131.

As shown in FIG. 4, the conveyance apparatus 10 selects one subject from a plurality of subjects receiving the conveyance service of the measurement device 20 provided by the conveyance system 1 (S1). The conveyance apparatus 10 acquires the history information of the selected subject from the history information table shown in FIG. 3 (S2). As described above, the history information includes the elapsed time since the subject last measured their biological information.

Based on the date and time information and the measurement presence/absence information included in the history information, the conveyance apparatus 10 identifies the past date and time when the subject measured their biological information using the measurement device 20, and determines whether it is necessary for the subject to measure their biological information (S3). For example, the conveyance apparatus 10 determines whether the period during which the subject has not measured their biological information (the aforementioned elapsed time) has exceeded a predetermined determination time.

If the conveyance apparatus 10 determines that it is not necessary for the subject to measure their biological information because the period during which the subject has not measured their biological information has not exceeded the predetermined determination time (NO at S3), it terminates this processing.

On the other hand, if the conveyance apparatus 10 determines that it is necessary for the subject to measure their biological information because the period during which the subject has not measured their biological information has exceeded the predetermined determination time (YES at S3), it identifies the current status of the subject (S4). For example, the conveyance apparatus 10 identifies the current status of the subject based on at least one of the captured image of the subject obtained by the camera 30 and the schedule information 133 including the pre-acquired schedule of the subject.

Based on the current status of the subject, the conveyance apparatus 10 determines whether measurement of the subject's biological information using the measurement device 20 is possible (S5). Specifically, the conveyance apparatus 10 determines whether the current status of the subject is an appropriate status for the subject to measure their biological information. For example, the conveyance apparatus 10 determines whether the current time is the subject's free time based on the subject's schedule information 133. Alternatively, by analyzing the subject's past behavior history based on the measurement presence/absence information, location information, status information, etc., included in the history information, the conveyance apparatus 10 determines whether the current status of the subject is a status with a high probability that the subject will measure their biological information using the measurement device 20.

The conveyance apparatus 10 may also determine whether the subject is in an appropriate situation to measure their biological information by recognizing the subject's actions and state through image recognition based on the captured image of the subject. For example, if the subject is in a meeting, in a conversation with others, at work, on the phone, or asleep, the conveyance apparatus 10 may determine that measurement of the subject's biological information using the measurement device 20 is not possible. Furthermore, if the subject is wearing headphones or earphones, the conveyance apparatus 10 may determine that measurement of the subject's biological information using the measurement device 20 is not possible.

If the conveyance apparatus 10 determines that measurement of the subject's biological information using the measurement device 20 is not possible (NO at S5), it terminates this processing.

On the other hand, if the conveyance apparatus 10 determines that measurement of the subject's biological information using the measurement device 20 is possible (YES at S5), it identifies the subject's location where the subject is located (S6). For example, the conveyance apparatus 10 identifies the subject's location based on the captured image of the subject obtained by the camera 30.

The conveyance apparatus 10 determines the behavior mode and speech sound for prompting the subject to measure their biological information using the measurement device 20 (S7). For example, by analyzing the subject's past behavior history based on the behavior mode information and speech script information included in the history information, the conveyance apparatus 10 determines the behavior mode and speech sound that would increase the probability of the subject measuring their biological information using the measurement device 20.

The conveyance apparatus 10 conveys the measurement device 20 to the subject (S8) and terminates this processing. When the conveyance apparatus 10 conveys the measurement device 20 to the subject, it acts according to the behavior mode determined in S7 and outputs the speech sound determined in S7. This prompts the subject to measure their biological information using the measurement device 20 conveyed by the conveyance apparatus 10.

As described above, when it is necessary to measure a subject's biological information, the conveyance apparatus 10 conveys the measurement device 20 to the subject's location, so the subject does not need to move to the installation location of the measurement device 20. Also, since the measurement device 20 conveyed by the conveyance apparatus 10 periodically appears in the subject's field of view, and the presence of the measurement device 20 is emphasized by the conveyance apparatus 10, it is possible to prompt the use of the measurement device 20 even to subjects who do not show interest in using the measurement device 20 or who have forgotten its existence. This makes it possible for the subject to continuously use the measurement device 20.

Furthermore, based on the subject's status, the conveyance apparatus 10 can convey the measurement device 20 to the subject at an appropriate time when the subject is able to measure their biological information. This can improve the probability that the subject will measure their biological information using the measurement device 20.

This allows the subject to continuously use the measurement device 20 to measure their own biological information, thereby grasping their health status leading up to a disease in a state where they have not yet become ill, and enabling them to take preventive measures. Even if a disease develops, the subject can grasp the progression of the disease by periodically measuring their biological information using the measurement device 20.

### Modification

Although the conveyance system 1 according to Embodiment 1 has been described above, the conveyance apparatus 10 is not limited to conveying the measurement device 20, and may convey conveyed objects other than the measurement device 20 that are used by a user. For example, the conveyed objects used by the user include items such as sweets, health foods, or supplements that can be purchased within the office. Also, the conveyed objects used by the user may include a questionnaire form for the user to fill out, or a PC displaying a questionnaire screen.

The conveyance apparatus 10 according to the modification may determine whether conveyance of the conveyed object to the user is possible based on the user's status, and if it is determined that conveyance of the conveyed object to the user is possible, it may identify the user's location where the user is located and convey the conveyed object to the user's location.

This allows the conveyance apparatus 10, when it determines that conveyance of a conveyed object to a user is possible, to convey the conveyed object to the user's location, eliminating the need for the user to move to the installation location of the conveyed object and prompting the use of the conveyed object even to users who are not interested in its use. Furthermore, the user can receive the conveyed object at an appropriate time, such as during their free time. This makes it possible for the user to continuously use the conveyed object.

### Embodiment 2

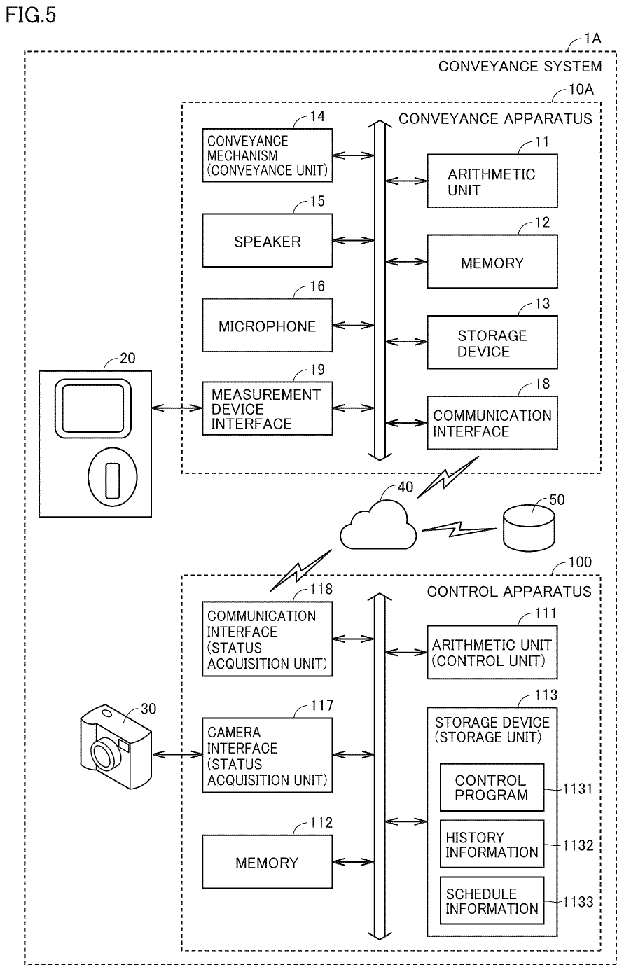

A conveyance system 1A according to Embodiment 2 will be described with reference to FIG. 5. Regarding the conveyance system 1A according to Embodiment 2, only the parts that differ from the conveyance system 1 according to Embodiment 1 will be described, and the description of the other parts will be omitted.

FIG. 5 is a diagram for explaining the configuration of the conveyance system 1A according to Embodiment 2. As shown in FIG. 5, the conveyance system 1A includes a conveyance apparatus 10A, a measurement device 20, a camera 30, and a control apparatus 100.

The control apparatus 100 is a server apparatus installed in an office or facility, or a cloud-based server apparatus. The control apparatus 100 includes an arithmetic unit 111, a memory 112, a storage device 113, a camera interface 117, and a communication interface 118.

The arithmetic unit 111 is an example of a “control unit” and is a computing entity (computer) that executes predetermined processing. The arithmetic unit 111 is composed of a processor such as a CPU, MPU, TPU, or GPU. It should be noted that a processor, which is an example of the arithmetic unit 111, has a function of executing predetermined processing by executing a predetermined program, but some or all of these functions may be implemented using dedicated hardware circuits such as an ASIC or an FPGA. “Processor” is not limited to a narrow sense processor that executes processing in a stored-program manner like a CPU, MPU, TPU, or GPU, but may include hard-wired circuits such as an ASIC or FPGA. The arithmetic unit 111 as described above can also be read as a processing circuitry. Note that the arithmetic unit 111 may be configured with a single chip or a plurality of chips.

The memory 112 includes a volatile storage area (e.g., a working area) that temporarily stores program codes or work memory when the arithmetic unit 111 executes various programs. Examples of the memory 112 include volatile memories such as DRAM and SRAM, or non-volatile memories such as ROM and flash memory.

The storage device 113 is an example of a “storage unit” and stores various programs executed by the arithmetic unit 111, various data, and the like. For example, the storage device 113 stores a control program 1131 executed by the arithmetic unit 111, and history information 1132 and schedule information 1133 used in processing according to the control program 1131. Furthermore, the storage device 113 stores the biological information of the subject measured using the measurement device 20. Specifically, the control apparatus 100 acquires biological information from the conveyance apparatus 10A and stores the acquired biological information of the subject. Note that the biological information of the subject acquired by the conveyance apparatus 10A may be output from the conveyance apparatus 10A to an external device 50 and stored by the external device 50. The storage device 113 may be one or more non-transitory computer readable media or one or more computer readable storage media. Examples of the storage device 113 include an HDD and an SSD.

The control program 1131 includes a program in which a processing procedure for the arithmetic unit 111 to execute processing for controlling the conveyance apparatus 10A is defined. By executing the processing according to the control program 1131, the arithmetic unit 111 can control the conveyance apparatus 10A to convey the measurement device 20 to a subject who needs to measure their biological information using the measurement device 20. The control program 1131 may be stored in the storage device 113 in advance, or may be acquired from the external device 50 by communicating with it.

Similar to the history information 132 shown in FIG. 2, the history information 1132 includes information regarding the measurement history of the subject's biological information. The history information 1132 is stored in the history information table shown in FIG. 3. Similar to the history information 132 shown in FIG. 2, the schedule information 1133 includes information regarding the subject's schedule. The history information 1132 and the schedule information 1133 may be stored in the storage device 113 in advance, or may be acquired from the external device 50 by communicating with it.

The camera interface 117 is an interface for communicating with the camera 30. The camera 30 includes the camera 30A installed in a room such as an office or facility, as shown in FIG. 1, and the camera 30B attached to the conveyance apparatus 10. The control apparatus 100 can acquire data of a captured image obtained by shooting with the camera 30 via the camera interface 117. The camera interface 117 can be an example of a “status acquisition unit” for the arithmetic unit 111 to acquire a captured image for identifying the subject's status.

The communication interface 118 is an interface for communicating with other devices via the network 40 through a wired or wireless connection. For example, the control apparatus 100 can control the conveyance apparatus 10A by communicating with the conveyance apparatus 10A via the communication interface 118 and the network 40. Also, the control apparatus 100 can acquire data such as the control program 1131, the history information 1132, and the schedule information 1133 by communicating with the external device 50 via the communication interface 118 and the network 40. The communication interface 118 can be an example of a “status acquisition unit” for the arithmetic unit 111 to acquire the schedule information 1133 for identifying the subject's status.

In the conveyance system 1A configured as described above, the control apparatus 100 executes processing similar to the processing shown in FIG. 4 based on the history information 1132. Specifically, the control apparatus 100 determines whether it is necessary to measure the subject's biological information, and if it determines that it is necessary, it identifies the subject's current status based on the subject's captured image or the schedule information 1133 including the pre-acquired schedule of the subject. The control apparatus 100 determines whether measurement of the subject's biological information using the measurement device 20 is possible based on the subject's current status. If the control apparatus 100 determines that measurement of the subject's biological information using the measurement device 20 is possible, it identifies the subject's location where the subject is located. Also, the control apparatus 100 determines the behavior mode and speech sound of the conveyance apparatus 10A that would increase the probability of the subject measuring their biological information using the measurement device 20. The control apparatus 100 transmits control information for moving the conveyance apparatus 10A to the subject's location, along with various information such as the identified subject's location, behavior mode, and speech sound, to the conveyance apparatus 10A. The conveyance apparatus 10A conveys the measurement device 20 to the subject according to the control information from the control apparatus 100.

The control apparatus 100 is also configured to execute other detailed processing included in the processing of FIG. 4 executed by the conveyance apparatus 10 of the conveyance system 1 according to Embodiment 1.

As described above, when it is necessary to measure a subject's biological information, the control apparatus 100 controls the conveyance apparatus 10 to convey the measurement device 20 to the subject's location, so the subject does not need to move to the installation location of the measurement device 20, and it is possible to prompt the use of the measurement device 20 even to subjects who are not interested in its use. This makes it possible for the subject to continuously use the measurement device 20.

Furthermore, based on the subject's status, the control apparatus 100 can control the conveyance apparatus 10A to convey the measurement device 20 at an appropriate time when the subject is able to measure their biological information. This can improve the probability that the subject will measure their biological information using the measurement device 20.

### Modification

Although the conveyance system 1A according to Embodiment 2 has been described above, the conveyance apparatus 10A is not limited to conveying the measurement device 20 to the subject's location, and may convey conveyed objects other than the measurement device 20 that are used by a user. The conveyed objects used by the user, similar to the modification of Embodiment 1, include items such as sweets, health foods, supplements available for purchase in the office, a questionnaire form for the user to answer, or a PC displaying a questionnaire screen.

The control apparatus 100 according to the modification may determine whether conveyance of the conveyed object to the user is possible based on the user's status, and if it is determined that conveyance of the conveyed object to the user is possible, it may identify the user's location where the user is located and control the conveyance apparatus 10A to convey the conveyed object to the user's location.

This allows the control apparatus 100, when it determines that conveyance of a conveyed object to a user is possible, to control the conveyance apparatus 10 to convey the conveyed object to the user's location, eliminating the need for the user to move to the installation location of the conveyed object and prompting the use of the conveyed object even to users who are not interested in its use. Furthermore, the user can receive the conveyed object at an appropriate time, such as during their free time. This makes it possible for the user to continuously use the conveyed object.

### Aspects

It will be understood by those skilled in the art that the plurality of exemplary embodiments described above are specific examples of the following aspects.
(Aspect 1) A conveyance system comprises a measurement device configured to measure biological information of a subject, a conveyance unit configured to convey the measurement device, a storage unit configured to store the biological information of the subject measured using the measurement device conveyed by the conveyance unit, a status acquisition unit configured to acquire a status of the subject, and a control unit configured to control the conveyance unit. The control unit determines whether measurement of the subject's biological information using the measurement device is possible based on the status of the subject acquired by the status acquisition unit, and when the measurement of the subject's biological information is possible, controls the conveyance unit to convey the measurement device to the subject.

According to the conveyance system of Aspect 1, it is possible to convey the measurement device to the subject at an appropriate time when the subject is able to measure their biological information, based on the status of the subject.
(Aspect 2) In the conveyance system according to Aspect 1, the status acquisition unit acquires schedule information of the subject as the status of the subject. The control unit determines whether the current time is a free time of the subject based on the schedule information, and when the current time is a free time, controls the conveyance unit to convey the measurement device to the subject.

According to the conveyance system of Aspect 2, it is possible to convey the measurement device to the subject at a time that is the subject's free time, based on the subject's schedule information.
(Aspect 3) In the conveyance system according to Aspect 1 or 2, the storage unit stores an elapsed time since the subject last measured their biological information. The control unit further determines whether measurement of the subject's biological information using the measurement device is possible based on the elapsed time.

According to the conveyance system of Aspect 3, it is possible to appropriately determine whether it is necessary to measure the user's biological information based on the elapsed time since the subject last measured their biological information.
(Aspect 4) In the conveyance system according to Aspect 3, the control unit determines whether the elapsed time has exceeded a determination time, and when the elapsed time has exceeded the determination time, controls the conveyance unit to convey the measurement device to the subject.

According to the conveyance system of Aspect 4, it is possible to have the user periodically measure their biological information.
(Aspect 5) In the conveyance system according to any one of Aspects 1 to 4, the status acquisition unit acquires a captured image of the subject as the status of the subject. The control unit determines whether the status of the subject is an appropriate status for measuring the subject's biological information based on the captured image, and when the status of the subject is an appropriate status, controls the conveyance unit to convey the measurement device to the subject.

According to the conveyance system of Aspect 5, it is possible to convey the measurement device to the subject at a time when the subject's status is an appropriate status for measuring the subject's biological information, based on the subject's captured image.
(Aspect 6) In the conveyance system according to any one of Aspects 1 to 5, the control unit determines that the status of the subject is not an appropriate status when the subject is wearing headphones or earphones.

According to the conveyance system of Aspect 6, when the subject is wearing headphones or earphones, it is possible to determine that the subject's status is not an appropriate status and not convey the measurement device to the subject.
(Aspect 7) In the conveyance system according to any one of Aspects 1 to 6, the conveyance unit outputs a sound for prompting the subject to measure their biological information using the measurement device.

According to the conveyance system of Aspect 7, it is possible to prompt the user to measure their biological information using sound.
(Aspect 8) In the conveyance system according to Aspect 7, the conveyance unit outputs a speech sound corresponding to the subject for prompting the subject to measure their biological information using the measurement device.

According to the conveyance system of Aspect 8, it is possible to prompt the user to measure their biological information using a speech sound.
(Aspect 9) In the conveyance system according to any one of Aspects 1 to 8, the conveyance unit performs an action corresponding to the subject for prompting the subject to measure their biological information using the measurement device.

According to the conveyance system of Aspect 9, it is possible to prompt the user to measure their biological information through action.
(Aspect 10) In the conveyance system according to any one of Aspects 1 to 9, the conveyance unit identifies the location of the subject based on at least one of a captured image of the subject, position information of the subject, and seat information set for the subject.

According to the conveyance system of Aspect 10, it is possible to appropriately identify the subject's location.
(Aspect 11) The conveyance system according to any one of Aspects 1 to 10 comprises a conveyance apparatus that conveys the measurement device. The conveyance apparatus includes the conveyance unit, the storage unit, the status acquisition unit, and the control unit.

According to the conveyance system of Aspect 11, the conveyance apparatus itself can determine whether the subject is able to measure their biological information based on the subject's status, and convey the measurement device to the subject at an appropriate time when the subject is able to measure their biological information.
(Aspect 12) The conveyance system according to any one of Aspects 1 to 10 comprises a conveyance apparatus that conveys the measurement device, and a control apparatus that controls the conveyance apparatus. The conveyance apparatus includes the conveyance unit. The control apparatus includes the storage unit, the status acquisition unit, and the control unit.

According to the conveyance system of Aspect 12, the conveyance apparatus can convey the measurement device to the subject at an appropriate time when the subject is able to measure their biological information, under the control of the control apparatus.
(Aspect 13) A conveyance system comprises a conveyance unit configured to convey a conveyed object used by a user, a status acquisition unit configured to acquire a status of the user, and a control unit configured to control the conveyance unit. The control unit determines whether conveyance of the conveyed object to the user is possible based on the status of the user acquired by the status acquisition unit, and when the conveyance of the conveyed object is possible, controls the conveyance unit to convey the conveyed object to the user.

According to the conveyance system of Aspect 13, it is possible to convey the conveyed object to the user at an appropriate time when the conveyance of the conveyed object is possible, based on the user's status.
(Aspect 14) The conveyance system according to Aspect 13 comprises a conveyance apparatus that conveys the conveyed object. The conveyance apparatus includes the conveyance unit, the status acquisition unit, and the control unit.

According to the conveyance system of Aspect 14, the conveyance apparatus itself can determine whether conveyance of the conveyed object is possible based on the user's status, and convey the conveyed object to the user at an appropriate time when the conveyance of the conveyed object is possible.
(Aspect 15) The conveyance system according to Aspect 13 comprises a conveyance apparatus that conveys the conveyed object, and a control apparatus that controls the conveyance apparatus. The conveyance apparatus includes the conveyance unit. The control apparatus includes the status acquisition unit and the control unit.

According to the conveyance system of Aspect 15, the conveyance apparatus can convey the conveyed object to the user at an appropriate time when the conveyance of the conveyed object is possible, under the control of the control apparatus.

The embodiments disclosed herein should be considered in all respects as illustrative and not restrictive. The scope of the present invention is indicated by the appended claims rather than by the foregoing description, and all changes which come within the meaning and range of equivalency of the claims are intended to be embraced therein.

### REFERENCE SIGNS LIST

1, 1A Conveyance system, 10, 10A Conveyance apparatus, 11, 111 Arithmetic unit, 12, 112 Memory, 13, 113 Storage device, 14 Conveyance mechanism, 15 Speaker, 16 Microphone, 17, 117 Camera interface, 18, 118 Communication interface, 19 Measurement device interface, 20 Measurement device, 30, 30A, 30B Camera, 40 Network, 50 External device, 100 Control apparatus, 131, 1131 Control program, 132, 1132 History information, 133, 1133 Schedule information.

What is claimed is:

1. A conveyance system, comprising:
a measurement device configured to measure biological information of a subject;
a conveyance unit configured to convey the measurement device;
a storage unit configured to store the biological information of the subject measured using the measurement device conveyed by the conveyance unit;
a status acquisition unit configured to acquire a status of the subject; and
a control unit configured to control the conveyance unit,
wherein the control unit is configured to:
determine whether measurement of the subject's biological information using the measurement device is possible based on the status of the subject acquired by the status acquisition unit, and
when the measurement of the subject's biological information is possible, control the conveyance unit to convey the measurement device to the subject.

2. The conveyance system according to claim 1, wherein
the status acquisition unit is configured to acquire schedule information of the subject as the status of the subject, and
the control unit is configured to:
determine whether a current time is a free time of the subject based on the schedule information, and
when the current time is the free time, control the conveyance unit to convey the measurement device to the subject.

3. The conveyance system according to claim 1, wherein
the storage unit is configured to store an elapsed time since the subject last measured biological information, and
the control unit is configured to further determine whether the measurement of the subject's biological information using the measurement device is possible based on the elapsed time.

4. The conveyance system according to claim 3, wherein the control unit is configured to:
determine whether the elapsed time has exceeded a determination time, and
when the elapsed time has exceeded the determination time, control the conveyance unit to convey the measurement device to the subject.

5. The conveyance system according to claim 1, wherein
the status acquisition unit is configured to acquire a captured image of the subject as the status of the subject, and
the control unit is configured to:
determine whether the status of the subject is an appropriate status for measuring the subject's biological information based on the captured image, and
when the status of the subject is the appropriate status, control the conveyance unit to convey the measurement device to the subject.

6. The conveyance system according to claim 5, wherein the control unit is configured to determine that the status of the subject is not the appropriate status when the subject is wearing headphones or earphones.

7. The conveyance system according to claim 1, wherein the conveyance unit is configured to output a sound for prompting the subject to measure biological information using the measurement device.

8. The conveyance system according to claim 7, wherein the conveyance unit is configured to output a speech sound corresponding to the subject for prompting the subject to measure biological information using the measurement device.

9. The conveyance system according to claim 1, wherein the conveyance unit is configured to perform an action corresponding to the subject for prompting the subject to measure biological information using the measurement device.

10. The conveyance system according to claim 1, wherein the conveyance unit is configured to identify a location of the subject based on at least one of a captured image of the subject, position information of the subject, and seat information set for the subject.

11. The conveyance system according to claim 1, comprising a conveyance apparatus configured to convey the measurement device,
wherein the conveyance apparatus includes the conveyance unit, the storage unit, the status acquisition unit, and the control unit.

12. The conveyance system according to claim 1, comprising:
a conveyance apparatus configured to convey the measurement device; and
a control apparatus configured to control the conveyance apparatus,
wherein the conveyance apparatus includes the conveyance unit, and
the control apparatus includes the storage unit, the status acquisition unit, and the control unit.

13. A conveyance system, comprising:
a conveyance unit configured to convey a conveyed object used by a user;
a status acquisition unit configured to acquire a status of the user; and
a control unit configured to control the conveyance unit,
wherein the control unit is configured to:
determine whether conveyance of the conveyed object to the user is possible based on the status of the user acquired by the status acquisition unit, and
when the conveyance of the conveyed object is possible, control the conveyance unit to convey the conveyed object to the user.

14. The conveyance system according to claim 13, comprising a conveyance apparatus configured to convey the conveyed object,
wherein the conveyance apparatus includes the conveyance unit, the status acquisition unit, and the control unit.

15. The conveyance system according to claim 13, comprising:
a conveyance apparatus configured to convey the conveyed object; and
a control apparatus configured to control the conveyance apparatus,
wherein the conveyance apparatus includes the conveyance unit, and
the control apparatus includes the status acquisition unit and the control unit.
